(19) United States
(12) Patent Application Publication
(10) Pub. No.: US 2021/0022902 A1
(43) Pub. Date: Jan. 28, 2021

(54) REAR SUPPORT ASSEMBLY OF NECK COLLAR

(71) Applicant: YU-CHIEN WANG, Taichung City (TW)

(72) Inventor: YU-CHIEN WANG, Taichung City (TW)

(21) Appl. No.: 16/856,672

(22) Filed: Apr. 23, 2020

(30) Foreign Application Priority Data
Jul. 24, 2019 (TW) 108126105

(51) Int. Cl. A61F 5/055

(52) U.S. Cl. CPC A61F 5/055

(57) ABSTRACT

A rear support assembly of neck collar is provided, including: a nape base, including a main body and two positioning mechanisms, each positioning mechanisms including a positioning member and an inserting space, the positioning member having a first combination portion; two support sheets, respectively movably inserted into the inserting space of one of the two positioning mechanisms, each said support sheets including a plurality of second combination portions, each second combination portions being engageable with the first combination portion of one of the two positioning mechanisms; one of the two support sheets and the main body respectively having one of a first guide track or a first rib; the other of the two support sheets and the main body respectively having one of a second guide track or a second rib.

### BACKGROUND OF THE INVENTION

### Field of the Invention

The present invention relates to a rear support assembly of neck collar.

### Description of the Prior Art

Neck is one of the most important parts of human body and also a part constantly to be moved. Once the neck is injured, it is easily to get injured again due to unexpected head and neck activities, which results in slow recovery. A neck protection device is developed to improve this phenomenon. The neck protection device is able to cover around the neck of a patient to support and restrict actions of the head and neck so as to avoid the neck getting injured again. The neck protection device disclosed in TWD159021 is the type described above.

However, the conventional rear support assembly of neck collar cannot be adjusted so that the conventional rear support assembly of neck collar may be too loose or too tight for the user. As a result, the conventional rear support assembly of neck collar lacks for the firmness and stability in wearing, which has a poor support.

The present invention is, therefore, arisen to obviate or at least mitigate the above mentioned disadvantages.

### SUMMARY OF THE INVENTION

An object of the present invention is to provide a rear support assembly of neck collar. Two support sheets are respectively stably positionably adjustable relatively to a main body.

To achieve the above and other objects, a rear support assembly of neck collar is provided, including: a nape base, including a main body, two positioning mechanisms, a plurality of openings, the two positioning mechanisms disposed separately on the main body in a first direction, each of the two positioning mechanisms including at least one stopper, a positioning member and an inserting space, the at least one stopper being connected with the main body and defining a distance and an inserting space therebetween, each said the positioning member and the at least one stopper of one of the two positioning mechanisms being disposed oppositely on the main body, each said positioning member having a first combination portion, each of the plurality of openings being configured for a band to be disposed therethrough; two support sheets, being configured to support a nape and a back of a human body respectively, being respectively movably inserted within the inserting space of one of the two positioning mechanisms, each of the two support sheets including a plurality of second combination portions, each of the plurality of second combination portions being adjustably engaged with the first combination portion of one of the two positioning mechanisms; at least one first guide track assembly, each of the at least one first guide track assembly including a first guide track and a first rib, the first guide track including a rail groove and at least one convex portion disposed within the rail groove, the first rib and the rail groove being adjustably engageable with each other, the first rib being engageable with the at least one convex portion; and at least one second guide track assembly, each of the at least one second guide track assembly disposed separately from at least one said first guide track assembly in the first direction, each of the at least one second guide track assembly including a second guide track and a second rib, the second rib and the second guide track being adjustably engageable with each other in the first direction; wherein one of the first guide track and the first rib is disposed on the main body, the other of first guide track and the first rib is disposed on one of the two support sheets; one of the second guide track and the second rib is disposed on the main body, the other of the second guide track and the second rib is disposed on the other of the two support sheets.

To achieve the above and other objects, a rear support assembly of neck collar is further provided, including: a nape base, including a main body, two positioning mechanisms, a plurality of openings, the two positioning mechanisms disposed separately on the main body in a first direction, each of the two positioning mechanisms including at least one stopper, a positioning member and an inserting space, the at least one stopper being connected with the main body and defining a distance and an inserting space therebetween, the positioning member of each of the two positioning mechanisms and the at least one stopper being disposed oppositely on the main body, the positioning member of each of the two positioning mechanisms having a first combination portion, each of the plurality of openings being configured for a band to be disposed therethrough; and two support sheets, being configured to support a nape and a back of a human body respectively, being respectively movably inserted within the inserting space of one of the two positioning mechanisms, each of the two support sheets including a plurality of second combination portions, each of the plurality of second combination portions being adjustably engageable with the first combination portion of one of the two positioning mechanisms, the two support sheets being movable to be at least partially overlapped with each other, one of the two support sheets having a first guide track, the other of the two support sheets including a first rib, the first guide track including a rail groove and at least one convex portion disposed within the rail groove, the first rib and the rail groove being adjustably engageable with each other, the first rib being engageable with the at least one convex portion.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a perspective view of a rear support assembly of neck collar according to a preferred embodiment of the present invention;

FIG. 2 is a breakdown drawing of the preferable embodiment of the present invention;

FIG. 3 is a partially-enlarged view of FIG. 2;

FIG. 4 is another breakdown drawing of the preferable embodiment of the present invention;

FIG. 4A is a partially-enlarged view of FIG. 4;

FIG. 5 is a partial cross-section view taken on line A-A of FIG. 1;

FIG. 6 is another partial cross-section view of the preferable embodiment of the present invention;

FIG. 7 is a partially-enlarged view of FIG. 6;

FIG. 8 is another partially-enlarged view of FIG. 6;

FIG. 9 is a perspective view of the rear support assembly of neck collar according to the preferred embodiment of the present invention in use;

FIG. 10 is partially cross-section view of another preferable embodiment of the present invention; and

FIG. 11 is a partially-enlarged view of FIG. 10.

### DETAILED DESCRIPTION OF THE PREFERRED EMBODIMENTS

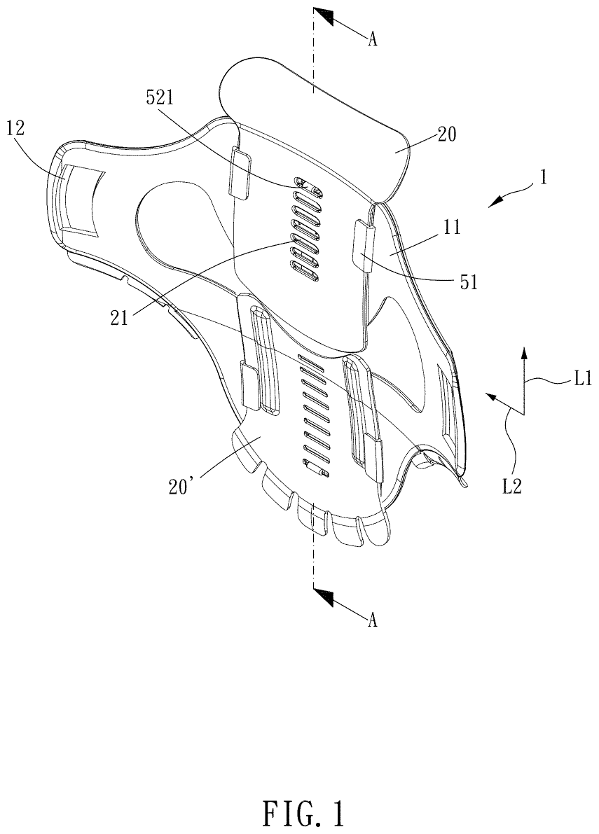

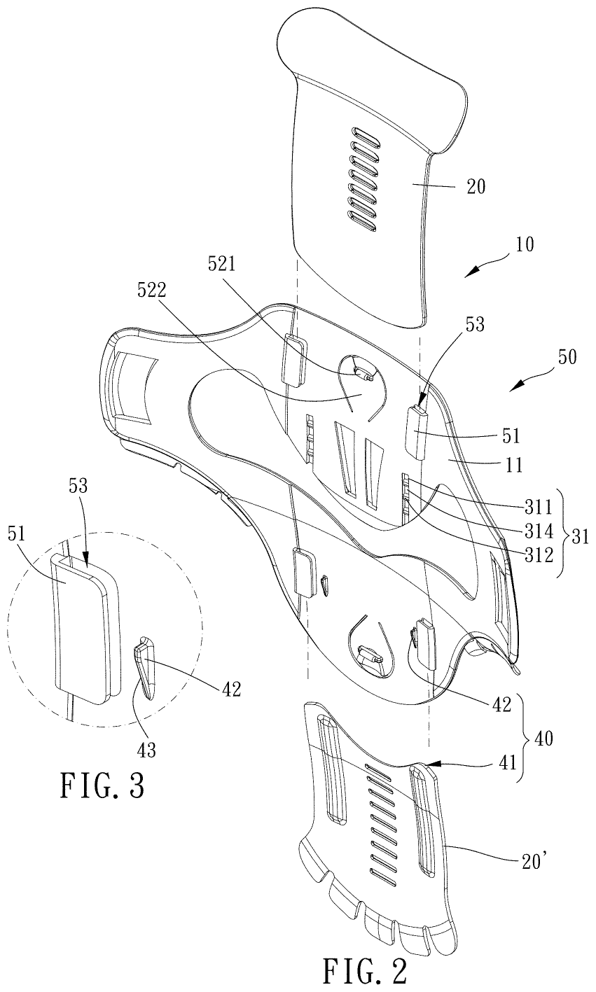

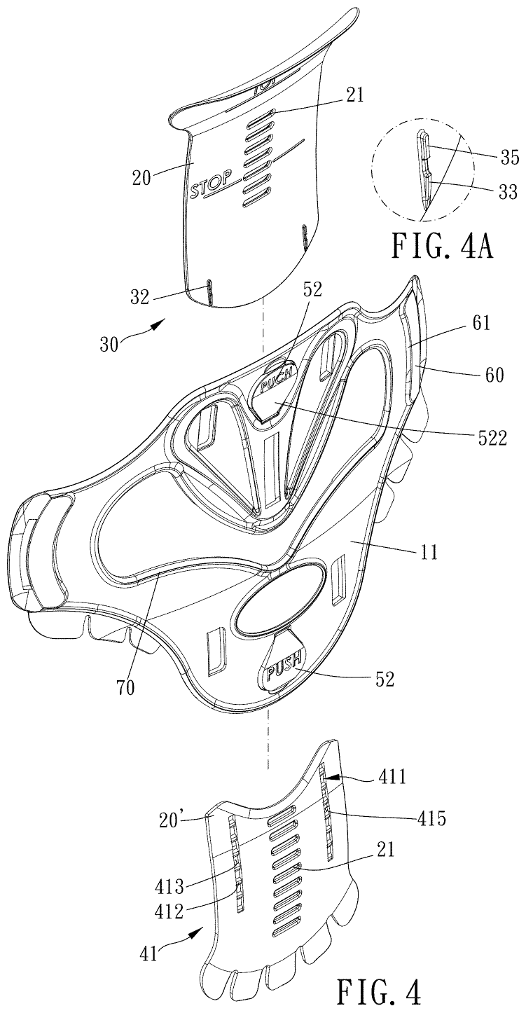

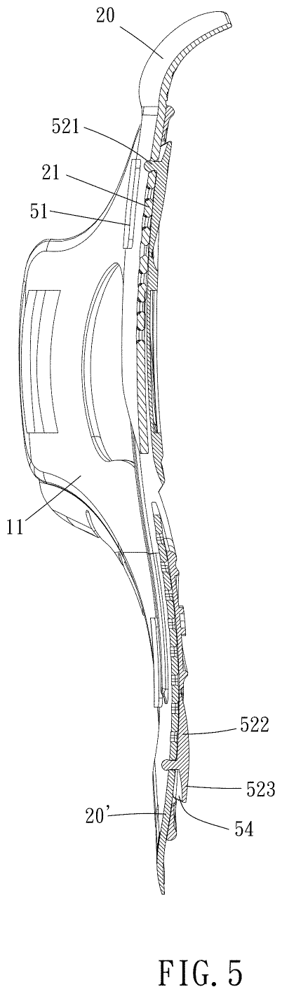

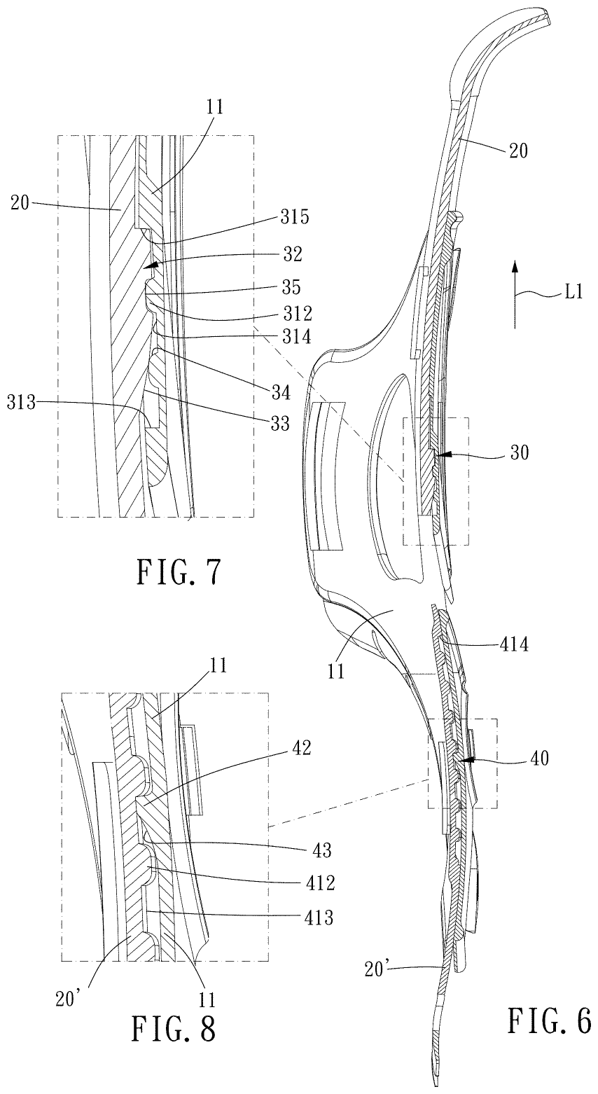

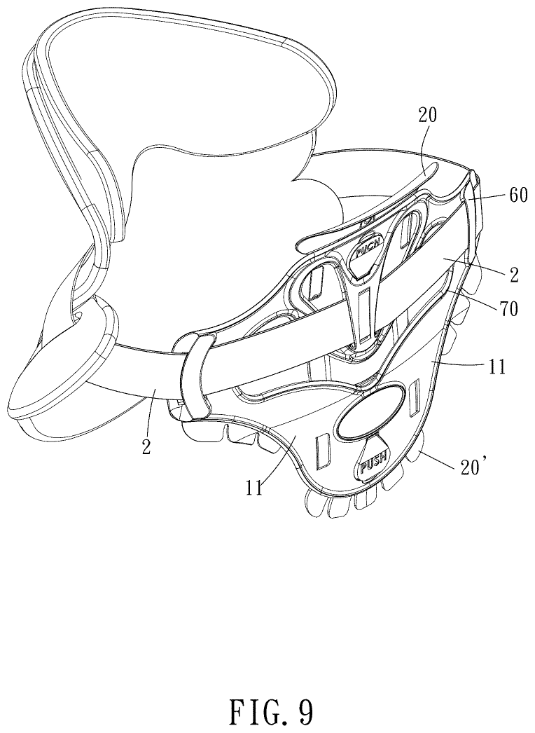

FIGS. 1-9 show a rear support assembly of neck collar according to a preferred embodiment of the present invention. The rear support assembly of neck collar includes a nape base 10, two support sheets 20, 20′, a first guide track assembly 30 and a second guide track assembly 40.

The nape base 10 includes a main body 11, two positioning mechanisms 50 and a plurality of openings 12. The two positioning mechanisms 50 is disposed separately on the main body 11 in a first direction L1. Each of the two positioning mechanisms 50 includes at least one stopper 51, a positioning member 52 and an inserting space 53. The at least one stopper 51 is connected with the main body 11 and defines a distance and an inserting space 53 therebetween. Each said positioning member 52 and the at least one stopper 51 of one of the two positioning mechanisms 50 are disposed oppositely on the main body 11. Each said positioning member 52 has a first combination portion 521. Each of the plurality of openings 12 is configured for a band 2 to be disposed therethrough. The support sheet 20 is configured to support a nape of a human body, the support sheet 20′ is configured to support a back of the human body. Each of the two support sheets 20, 20′ is respectively movably inserted within the inserting space 53 of one of the two positioning mechanisms 50. Each of the two support sheets 20, 20′ includes a plurality of second combination portions 21. Each of the plurality of second combination portions 21 is adjustably engaged with the first combination portion 521 of one of the two positioning mechanisms 50.

The at least one first guide track assembly 30 includes a first guide track 31 and a first rib 32. The first guide track 31 includes a rail groove 311 and at least one convex portion 312 disposed within the rail groove 311, the first rib 32 and the rail groove 311 is adjustably engageable with each other. The first rib 32 is engageable with the at least one convex portion 312. Specifically, the first rib 32 does not get stuck with the at least one convex portion 312, so as to provide a multi-stage adjustment. One of the first guide track 31 and the first rib 32 is disposed on the main body 11, the other of the first guide track 31 and the first rib 32 is disposed on the support sheet 20. In this embodiment, the first guide track 31 is disposed on the main body 11, and the first rib 32 is disposed on the support sheet 20.

The second guide track assembly 40 is disposed separately from at least one said first guide track assembly 30 in the first direction L1. Each of the at least one second guide track assembly 40 includes a second guide track 41 and a second rib 42. The second rib 42 and the second guide track 41 are adjustably engageable with each other in the first direction L1. One of the second guide track 41 and the second rib 42 is disposed on the main body 11, the other of the second guide track 41 and the second rib 42 is disposed on the support sheet 20′. In this embodiment, the second rib 42 is disposed on the main body 11, and the second guide track 41 is disposed on the support sheet 20′. In summary, each of the two support sheets 20, 20′ is smoothly adjustable relatively to the main body 11 and may not be rotated relatively to the main body 11. Moreover, it is easy to know an adjustment range of each of the two support sheets 20, 20′, and each of the two support sheets 20, 20′ can be fast positioned and released. The two support sheets 20, 20′ are respectively made of flexible elastic material, so as to be easy bent and deformed.

Specifically, at least part of each said the positioning member 52 is movable to a blocking position and a releasing position, each said the first combination portion 52 is insertable into the inserting space 53 when changing in position. Each said first combination portion 521 is located at the inserting space 53 and engaged with one of the plurality of second combination portions 21 of one of the two support sheets 20, 20′ when each said the positioning member 52 is located at the blocking position; each said the first combination portion 52 is departed from one of the plurality of second combination portions 21 of one of the two support sheets 20, 20′ and moved away from one of the two support sheets 20, 20′ when each said the positioning member 52 is located at the releasing position, so that each of the two support sheets 20, 20′ is adjustable. Each said positioning member 52 includes a base portion 522 and a pull portion 523. The base portion 522 is made of a flexible elastic material. Each said first combination portion 521 is disposed on the base portion 522. When being stressed by an external force, the base portion 522 is bent relatively to the main body 11 and deformed; when being unstressed, the base portion 522 elastically recovers to an initial position. Each said pull portion 523 is protuberantly connected with the base portion 522 and defines an interval 54 with the main body 11, each said interval 54 is configured for insertion of a finger. Each said interval 54 open toward at two opposite sides of the main body 11, so that the positioning member 52 can be easy bent. A connection relationship of the positioning member 52 and the support sheet 20′ is the same as a connection relationship of the positioning member 52 and the support sheet 20.

The at least one first guide track assembly 30 includes two said first guide track assemblies 30. The two said first guide track assembly 30 are arranged separately from each other in a second direction L2 transverse to the first direction L1, and the positioning member 52 of one of the two positioning mechanisms 50 is disposed between the two said first guide track assemblies 30. In this embodiment, the second guide track assembly 40 includes two said second guide track assemblies 40. An arrangement of two said second guide track assemblies 40 is the same as an arrangement of two said first guide track assembly 30, so as to improve an adjustment stability of each of the two support sheets 20, 20′.

Each said second guide track 41 is the same as the first track 31 and also includes a rail groove 411 and a plurality of convex portion 412 disposed within the rail groove 411, a number and function of the plurality of convex portions 412 and a number and function of the plurality of second combination portions 21 are the same.

A top end of each of the at least one convex portion 312, 412 of the first guide track 31 and the second guide track 41 is lower than an opening 313, 415 of the rail groove 311, 411. Thus, the first rib 32 and the second rib 42 can respectively constantly be inserted into the first guide track 31 or the second guide track 41.

Each said first guide track 31 includes a plurality of said convex portions 312 and a plurality of concaves 314 each disposed between adjacent two of the plurality of said convex portions 312. Each said first rib 32 is disengageably engaged within one of the plurality of concaves 314 and abutted against at least one of said plurality of convex portion 312. When the support sheet 20 is adjusted, the first rib 32 is abutted against the at least one convex portion 312 to be disengaged from one of the plurality of concaves 314. Each of the two second guide tracks 41 has a plurality of concaves 413. A number and function of the plurality of concaves 413 and a number and function of the concaves 314 are the same.

The first rib 32 has a guide inclined surface 33 which is inclined in the first direction L1, and the at least one convex portion 312 is abutable against the guide inclined surface 33. Preferably, each of the plurality of convex portions 312 has two arc-shaped corner 34 which are disposed on two opposite sides thereof in the first direction L1, so that the first rib 32 can easy depart from and not get stuck with each of the plurality of concaves 314. The second rib 42 has a guide inclined surface 43 which is the same as the guide inclined surface 33.

The first rib 32 has a recess 35, a depth of the recess 35 is less than a maximum thickness in a direction in which the first rib 32 protrudes, and the recess 35 and the at least one convex portion 312 are disengageably engaged with each other. The recess 35 and the at least one convex portion 312 are smoothly engaged and disengaged with each other. The rail groove 311 faces the support sheet 20. Therefore, the first rib 32 can be easy moved toward or far away from the rail groove 311.

Each said rail groove 311 has an abutting surface facing 315 in the first direction L1, and each said abutting surface 315 is abutable against each said first rib 32 in the first direction L1, so as to prevent the support sheet 20 from being disengaged from the main body 11. In this embodiment, the rail groove 411 has an abutting surface 414 which has the same structure and function as each said abutting surface 315.

The main body 11 has two block walls 60, and each of the two block walls 60 is connected with the main body 11 and defines a through hole 61 therebetween, and the through hole 61 is configured for the band 2 to be disposed therethrough. The main body 11 has a heart-shaped convex portion 70, and the heart-shaped convex portion 70 and each of the two block walls 60 are configured to be disposed on two opposite sides of the band 2, so as to be adapted for stably supporting the band 2.

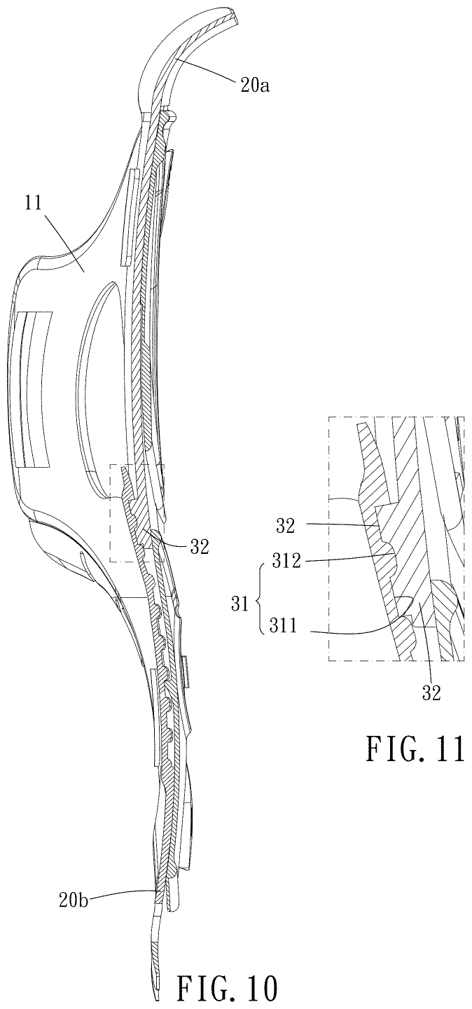

FIG. 10 and FIG. 11 show another preferred embodiment of the present invention, the two support sheets 20a, 20b are movable to be at least partially overlapped with each other, one of the two support sheets 20a, 20b includes the first guide track 31, the other of the two support sheets 20a, 20b includes the first rib 32, the first guide track 31 includes the rail groove 311 and at least one convex portion 312 disposed within the rail groove 311. The first rib 32 is adjustably engageable within the rail groove 311 and engageable with the at least one of the convex portion 312, the two support sheets 20a, 20b are not easy flipped over when being stressed by the external force.

In summary, the rear support assembly of neck collar emphasizes wide applicability. The two support sheets are respectively slidable and positionable relative to nape base to apply to the neck of different users, and the two support sheets can perfectly support and protect the neck to prevent the neck from getting injured again due to unexpected movement. Moreover, the first and the second guide track assemblies can apply the two support sheets to be stably adjusted.

Although particular embodiments of the invention have been described in detail for purposes of illustration, various modifications and enhancements may be made without departing from the spirit and scope of the invention. Accordingly, the invention is not to be limited except as by the appended claims.

What is claimed is:

1. A rear support assembly of neck collar, including:
a nape base, including a main body, two positioning mechanisms, a plurality of openings, the two positioning mechanisms disposed separately on the main body in a first direction, each of the two positioning mechanisms including at least one stopper, a positioning member and an inserting space, the at least one stopper being connected with the main body and defining a distance and an inserting space therebetween, each said the positioning member and the at least one stopper of one of the two positioning mechanisms being disposed oppositely on the main body, each said positioning member having a first combination portion, each of the plurality of openings being configured for a band to be disposed therethrough;
two support sheets, being configured to support a nape and a back of a human body respectively, being respectively movably inserted within the inserting space of one of the two positioning mechanisms, each of the two support sheets including a plurality of second combination portions, each of the plurality of second combination portions being adjustably engaged with the first combination portion of one of the two positioning mechanisms;
at least one first guide track assembly, each of the at least one first guide track assembly including a first guide track and a first rib, the first guide track including a rail groove and at least one convex portion disposed within the rail groove, the first rib and the rail groove being adjustably engageable with each other, the first rib being engageable with the at least one convex portion; and
at least one second guide track assembly, each of the at least one second guide track assembly disposed separately from at least one said first guide track assembly in the first direction, each of the at least one second guide track assembly including a second guide track and a second rib, the second rib and the second guide track being adjustably engageable with each other in the first direction;
wherein one of the first guide track and the first rib is disposed on the main body, the other of first guide track and the first rib is disposed on one of the two support sheets; one of the second guide track and the second rib is disposed on the main body, the other of the second guide track and the second rib is disposed on the other of the two support sheets.

2. A rear support assembly of neck collar, including:
a nape base, including a main body, two positioning mechanisms, a plurality of openings, the two positioning mechanisms disposed separately on the main body in a first direction, each of the two positioning mechanisms including at least one stopper, a positioning member and an inserting space, the at least one stopper being connected with the main body and defining a distance and an inserting space therebetween, the positioning member of each of the two positioning mechanisms and the at least one stopper being disposed oppositely on the main body, the positioning member of each of the two positioning mechanisms having a first combination portion, each of the plurality of openings being configured for a band to be disposed therethrough; and
two support sheets, being configured to support a nape and a back of a human body respectively, being respectively movably inserted within the inserting space of one of the two positioning mechanisms, each of the two support sheets including a plurality of second combination portions, each of the plurality of second combination portions being adjustably engageable with the first combination portion of one of the two positioning mechanisms, the two support sheets being movable to be at least partially overlapped with each other, one of the two support sheets having a first guide track, the other of the two support sheets including a first rib, the first guide track including a rail groove and at least one convex portion disposed within the rail groove, the first rib and the rail groove being adjustably engageable with each other, the first rib being engageable with the at least one convex portion.

3. The rear support assembly of neck collar of claim 1, wherein the at least one first guide track assembly includes two said first guide track assemblies, the two said first guide track assemblies are arranged separately from each other in a second direction transverse to the first direction, and the positioning member of one of the two positioning mechanisms is disposed between the two said first guide track assemblies.

4. The rear support assembly of neck collar of claim 1, wherein a top end of the at least one convex portion is lower than an opening of the rail groove.

5. The rear support assembly of neck collar of claim 1, wherein each said first guide track includes a plurality of said convex portions and a plurality of concaves each disposed between adjacent two of the plurality of said convex portions, and each said first rib is disengageably engaged within one of the plurality of concaves and abutted against at least one of said plurality of convex portions.

6. The rear support assembly of neck collar of claim 1, wherein the first rib has a guide inclined surface which is inclined in the first direction, and the at least one convex portion is abutable against the guide inclined surface.

7. The rear support assembly of neck collar of claim 1, wherein the rail groove faces the support sheet.

8. The rear support assembly of neck collar of claim 1, wherein the first rib has a recess, a depth of the recess is less than a maximum thickness in a direction in which the first rib protrudes, and the recess and the at least one convex portion are disengageably engaged with each other.

9. The rear support assembly of neck collar of claim 1, wherein at least part of each said positioning member is movable to a blocking position and a releasing position, each said the first combination portion is insertable into the inserting space when changing in position, wherein each said first combination portion is located at the inserting space and engaged with one of the plurality of second combination portions of one of the two support sheets when each said the positioning member is located at the blocking position; each said the first combination portion is departed from one of the plurality of second combination portions of one of the two support sheets and moved away from one of the two support sheets when each said the positioning member is located at the releasing position; each said positioning member includes a base portion and a pull portion, the base portion is made of a flexible elastic material, each said first combination portion is disposed on the base portion, each said pull portion is protuberantly connected with the base portion and defines an interval with the main body; and each said interval open toward at two opposite sides of the main body.

10. The rear support assembly of neck collar of claim 3, wherein each said first guide track includes a plurality of said convex portions and a plurality of concaves each disposed between adjacent two of the plurality of said convex portions, and each said first rib is disengageably engaged within one of the plurality of concaves and abutted against at least one of said plurality of convex portions; the first rib has a guide inclined surface which is inclined in the first direction, and the at least one convex portion is abutable against the guide inclined surface; each of the plurality of convex portions has two arc-shaped corners which are disposed on two opposite sides thereof in the first direction; the first rib has a recess, a depth of the recess is less than a maximum thickness in a direction in which the first rib protrudes, and the recess and the at least one convex portion are disengageably engaged with each other; at least part of each said positioning member is movable to a blocking position and a releasing position, each said the first combination portion is insertable into the inserting space when changing in position, wherein each said first combination portion is located at the inserting space and engaged with one of the plurality of second combination portions of one of the two support sheets when each said the positioning member is located at the blocking position; each said the first combination portion is departed from one of the plurality of second combination portions of one of the two support sheets and moved away from one of the two support sheets when each said the positioning member is located at the releasing position; each said positioning member includes a base portion and a pull portion, the base portion is made of a flexible elastic material, each said first combination portion is disposed on the base portion, each said pull portion is protuberantly connected with the base portion and defines an interval with the main body; and each said interval open toward at two opposite sides of the main body, each said interval is configured for insertion of a finger; each said rail groove has an abutting surface facing in the first direction, and each said abutting surface is abutable against each said first rib in the first direction; the main body has two block walls disposed separately from each other, and each of the two block walls is connected with the main body and defines a through hole therebetween, and the through hole is configured for the band to be disposed therethrough; the main body has a heart-shaped convex portion, and the heart-shaped convex portion and each of the two block walls are configured to be disposed on two opposite sides of the band.
